(19) United States
(12) Patent Application Publication
(10) Pub. No.: US 2022/0047404 A1
(43) Pub. Date: Feb. 17, 2022

(54) METHOD FOR MANUFACTURING PORTIONS OF A PROSTHETIC SHAFT AND KIT

(71) Applicant: Romedis GMBH, Neubeuern (DE)

(72) Inventor: Andreas RADSPIELER, Neubeuern (DE)

(21) Appl. No.: 17/289,597

(22) PCT Filed: Nov. 12, 2019
(86) PCT No.: PCT/EP2019/081071

§ 371 Date: Apr. 28, 2021

(30) Foreign Application Priority Data
Nov. 12, 2018 (DE) 10 2018 128 231.1
Jan. 25, 2019 (DE) 102019 101 895.1

(51) Int. Cl. A61F 2/50; A61F 2/80; G16H 10/60

(52) U.S. Cl. CPC A61F 2/5046; A61F 2/80; A61F 2002/5047; A61F 2002/505; G16H 10/60

(57) ABSTRACT

The present invention relates to a method for manufacturing or planning the manufacturing of a prosthetic shaft, an inner or outer shaft and/or of an extension of the prosthetic shaft, wherein the prosthetic shaft is provided for receiving a limb stump of a patient P. The present invention further relates to prosthetic shaft and a kit. Furthermore, a computer system, a digital storage medium, a computer program product and a computer program are proposed.

The present invention relates to a method for manufacturing a prosthetic shaft or for the planning of manufacturing a prosthetic shaft according to claim 1. The present invention further relates to a prosthetic shaft according to claim 11 and to a kit according to claim 12. Furthermore, a computer system according to claim 14, a digital storage medium according to claim 15, a computer program product according to claim 16 and a computer program according to claim 17 are proposed.

Leg amputees may regain mobility using leg prostheses. Modern leg prostheses include various modules (prosthesis shaft, knee, lower leg and foot modules), which may be combined to meet the various needs of the prosthesis wearer (hereinafter referred to in short as wearer) in terms of fundamental mobility, sport activities and aesthetic perceptions.

The present invention relates to a method for manufacturing or planning the manufacturing or creating of a prosthetic shaft of a prosthesis for, e.g., the lower extremities, e.g., for a leg prosthesis. The prosthetic shaft is the module of the prosthesis, which represents the connection between the mechanical replacement of the extremity and the residual limb stump (in short also referred to as stump) of the prosthesis wearer, e.g. a thigh stump.

The prosthetic shaft is connected at its distal end (that end facing away from the prosthesis wearer) to a mechanical extremity replacement, for example, in the case of the lower extremity, a modular knee joint-lower leg-foot device for the thigh amputee or a modular lower leg-foot device for the lower-leg amputee. At its proximal end (that end facing the prosthesis wearer), the stump is inserted into the prosthetic shaft. The prosthetic shaft should sit tightly and as positively as possible on the stump. The precise fit of the connection with the limb stump determines how securely the prosthesis is held on the stump.

In order to transmit the partly considerable forces between the wearer's body or stump on the one hand and the prosthesis on the other, which occur when standing, walking, standing up, running, etc., a high degree of strength or rigidity of the prosthesis shaft is required. This is ensured by a correspondingly stiff outer section of the prosthetic shaft, the so-called outer shaft. However, since its stiffness causes pressure on the stump, which is regularly perceived as uncomfortable or even painful and can lead to pressure points, the outer shaft is supplemented by a so-called inner shaft (also known as a sleeve) on its inside to increase wearing comfort. The inner shaft usually comprises an elastic material. It may consist of this in the form of an elastic sleeve (liner) or be padded with it.

The object of the present invention may be to optimize the manufacturing of a prosthetic shaft or its planning. Furthermore, a prosthetic and a kit, a computer system, a digital storage medium, a computer program product and a computer program are to be proposed.

The object according to the present invention can be achieved by a method for manufacturing or planning a prosthetic shaft with the features of claim 1. Furthermore, the object may be achieved by a prosthetic shaft according to claim 11 and a kit according to claim 12. Furthermore, a computer system with the features of claim 14, a digital storage medium with the features of claim 15, a computer program product with the features of claim 16 and a computer program with the features of claim 17 contribute to achieving the object according to the present invention.

According to the present invention, generally a method for manufacturing or for planning the manufacturing of a prosthetic shaft, an inner and/or an outer shaft and/or an extension of the prosthetic shaft is therefore proposed. Thereby prosthetic shaft is provided in order to receive a limb stump.

The method according to the present invention comprises determining data, in particular geometric data, or providing data, in particular geometric data. In this, preferably such data, in particular geometric data, is determined which will determine or co-determine the shape of the limb stump at at least one point in time in the future. Preferably, data is determined here which, at least at the first wearing time, will co-determine or determine the shape of the prosthetic shaft, the inner or outer shaft or the extension (or the shape which these should then have). The data or geometric data may be estimated values or expected values.

The method optionally further comprises either creating the prosthetic shaft or sections thereof. This is done based on the determined data and/or geometric data. Alternatively or in addition to the aforementioned creating of the prosthetic shaft or sections thereof, the method encompasses creating at least one control signal, in particular creating a control file with control signals, based on which a manufacturing machine may (e.g. directly or indirectly) perform steps for creating the prosthetic shaft, the inner or outer shaft or the extension and/or create at least one of the aforementioned devices or parts thereof.

Thereby, the time of creating (also: creating time) is earlier than the first wearing time.

The method in its general or most general embodiment optionally has no further features.

The prosthetic shaft, inner shaft or outer shaft according to the present invention, or the extension according to the present invention for the prosthetic shaft are manufactured by or using the method according to the present invention.

A kit with at least two elements of a group is also proposed by the present invention. The group consists of a prosthetic shaft, an inner shaft, an outer shaft and at least one extension for the prosthetic shaft. In this, all elements of the groups are created based on the method according to the present invention, that is, e.g. manufactured or produced or created.

A computer system which is programmed to perform the method according to the present invention is proposed by the present invention. It may for this purpose comprise a programmable data processing device.

A particularly digital, particularly non-volatile storage medium (herein denoted also as carrier), according to the present invention, in particular in the form of a floppy disk, RAM, ROM, CD, hard disk, DVD, USB stick, flash card, SD card, FeRAM or EPROM, in particular with electronically or optically readable control signals, may be configured such that to configure a control device into a control device with which the method according to the present invention described herein may be effected.

In this, all, several or some of the machine-induced method steps may be prompted.

A computer program product according to the present invention comprises a program code that is volatile or saved on a machine-readable carrier, through which a control device is configured such that the method according to the present invention described herein may be effected.

In this, all, several or some of the machine-induced method steps may in turn be prompted.

The term “machine-readable carrier” as used herein, refers in certain embodiments of the present invention to a carrier, which contains data or information interpretable by software and/or hardware. The carrier may be a data carrier, such as a floppy disk, a CD, DVD, a USB stick, a flashcard, an SD card, an FeRAM, an EPROM or the like.

A computer program product may according to the present invention be understood as, for example, a computer program which is stored on a carrier, an embedded system as a comprehensive system with a computer program (for example, an electronic device with a computer program), a network of computer-implemented computer programs (for example, a client-server system, a cloud computing system, etc.) or a computer on which a computer program is loaded, running, saved, executed or developed.

A computer program according to the present invention encompasses a program code by which a control device is configured such that the method according to the present invention described herein may be effected.

In this, all, several or some of the machine-induced method steps may be prompted.

According to the present invention, a computer program may be understood to mean, for example, a physical, marketable software product which comprises a program.

In all the following statements, the use of the expression “may be” or “may have” and so on, is to be understood synonymously with “preferably is” or “preferably has,” and so on respectively, and is intended to illustrate embodiments according to the present invention.

Whenever numerical words are mentioned herein, the person skilled in the art shall recognize or understand them as indications of numerical lower limits. Unless it leads the person skilled in the art to an evident contradiction, the person skilled in the art shall comprehend for example the specification of “one” as encompassing “at least one”. This understanding is also equally encompassed by the present invention as the interpretation that a numerical word, for example, “one” may alternatively mean “exactly one”, wherever this is evidently technically possible for the person skilled in the art. Both understandings are encompassed by the present invention and apply to all numerical words used herein.

Whenever “programmed” or “configured” is mentioned herein, it is then disclosed that these terms are interchangeable.

Whenever an applicability or a method step is mentioned herein, the present invention additionally encompasses also a corresponding programming or configuring of a suitable apparatus or a section thereof, for example, of a computer system, and devices programmed in such a way. The apparatuses may each be named after the method step they carry out.

Advantageous developments of the present invention are each subject-matter of the dependent claims and embodiments.

Whenever an embodiment is mentioned herein, it is then an exemplary embodiment according to the present invention.

Embodiments according to the present invention may comprise one or more of the following features in any combination, unless the person skilled in the art recognizes a specific combination as technically impossible. Also, the subject-matters of the dependent claims specify embodiments according to the present invention.

When the term creating is mentioned herein, it can be understood to mean, when referring to physical objects such as the prosthetic shaft, the inner shaft, etc., as manufacturing, making, fabricating or producing the same. The creation time is then the time of manufacturing, making, fabricating or producing.

In some embodiments, the data is not geometric data, although it may herein nevertheless be referred to as such.

In some embodiments, the data is topographic data of the limb stump.

In several embodiments, the data or geometric data, determined in the method according to the present invention, is predicted data. It is then not actual data and/or measured data representing the shape of the limb stump at the time the data is determined or at the time the prosthetic shaft or control signals are created. However, such measured data may have been used for prediction. The predicted data may be or may comprise variable dimensions of the limb stump and/or data which is subject to changes over time, for example post-operatively or as a result of a previous surgical operation.

The circumference or the water content (edema) of the limb stump is mentioned here as an example.

In some embodiments of the method according to the present invention, determining the data and/or geometric data, which is predicted data, takes into account patient data reflecting the, in particular current, health condition of the patient. The patient data may reflect the health condition at the moment of determining or creating. They may, in addition or alternatively, provide information about medical characteristics that were present prior to or at the time of determination, such as pre-existing diseases. The patient data may be measured and/or anamnestically or clinically collected, etc.

Examples of this patient data may include:
the patient's age,
information on the time elapsed since the amputation,
whether the fitting of the patient with a prosthesis is a fitting of a first or of a subsequent prosthesis,
whether the patient is obese (measurable, for example, by body mass index (BMI) or bioimpedance, e.g., by an electronic scale, body composition monitor, and the like),
the genesis or cause of the amputation (for example, as a result of peripheral arterial occlusive disease (PAOD), of a trauma or inflammation and/or the presence of a tumor, inflammation and/or the like),
whether the patient has a peripheral arterial occlusive disease,
cardiac performance data (for example, whether the patient has heart failure),
whether the patient has a protein deficiency,
whether the patient suffers from diabetes,
whether there is an insufficiency of the patient's lymphatic system (for example, pre- or postoperative lymphedema),
the patient's venous situation (for example, the presence of deep vein thrombosis (DVT) or other thromboses and/or existing varicose veins (varices) and/or whether the patient has already undergone varicose vein surgery, for example),
operations already performed (in particular in the area of the extremity affected by the amputation, e.g. in the groin due to a cardiac catheter operation),
heart failure,
metabolic diseases,
movement behavior of the patient (athlete, walker, office worker without sport balance, etc.),
composition of body tissue, e.g. of the limb stump, by regarding the fluid, fat and/or muscle content (e.g. by a bio-impedance measurement, e.g. measured by a body composition monitor) or information about a content thereof (in absolute values), information about the blood circulation, information about the distance over which the limb stump protrudes over bony structures (e.g. femur), e.g. in their longitudinal direction,
medication of the patient, in particular long-term medication.

In this, the data may be used and/or processed individually or in any combination to predict changes in the shape of the limb stump over time (e.g. between the time of creation and the first wearing time) and thus to determine the geometric data. In particular, grading of findings or staging of the aforementioned findings and other pre-existing diseases may be taken into account and included in the prognosis.

The measure with which such patient data enters into the determination of the geometric data may be an input or information in percent, centimeters, or in another manner or—purely optionally—in other units. This measure may lead to a change in the prognosis data determined, for example, from a collective of patients, as exemplarily shown in FIG. 6, or may be incorporated into them, for example, by subtracting empirical values known from observation of diabetics or patients with other medical conditions relevant to future changes in the shape of the limb stump (such as those mentioned, for example, above). Likewise, such a measure may, purely optionally, need to be added to the collective data for some specific medical conditions. The present invention also encompasses a percentage factor (or its use) by which the empirical data of the collective of patients whose patients do not have the specific medical condition can be multiplied.

According to the present invention, the collective data may have been collected from patients who are not affected by the concrete medical condition. This data may be adapted to the specific patient with the specific medical condition as described above, e.g. by using the above measure.

However, in some embodiments, the collective data has already been collected from patients who have the one or the other of the above-mentioned medical conditions or another medical condition, or those who have any combinations of two or more such medical conditions. In this case, no adjustment is necessary; the prognosis data collected in the group, generally do not need to be adapted to the medical condition, since this was also present in the collective. However, an adjustment may still be made optionally, either because it may be necessary or advantageous. For example, it may be advantageous to optionally consider the degree to which the medical condition is or was present (staging, stage, degree, classification, NYHA, age, etc.) for fine adjustment.

The patient data may be stored, for example in one of the devices according to the present invention, or in a location that can be accessed by the devices according to the present invention, such as in a database, etc. The devices according to the present invention may be configured to (re)access such stored data. Alternatively, this data can be entered by the user of the devices according to the present invention by an input device provided and/or configured for this purpose. The optionally required comparisons between patient data of the specific patient and a collective, if provided, may optionally run automatically, for example once, several times and/or regularly.

In some embodiments of the method according to the present invention, the patient data is taken into account when determining the data or geometric data, e.g., by mathematical calculation operations.

If reference is made herein to mathematical calculation operations, such as addition, subtraction, division or multiplication, all other known mathematical calculation operations are also encompassed by the present invention. The ways mentioned above and below for determining the prognosis data or for determining or adapting the geometric data, in particular due to medical conditions, are purely exemplary.

In some embodiments, the data or geometric data determined in the method according to the present invention is or comprises the result of an estimation. Alternatively or in addition, they are read from a reference source. Such a reference source may be, for example, a table of values and/or a database.

In several embodiments, this data is or comprises the result of a calculation based on an algorithm. This algorithm may in turn be created by expert systems and/or may be based on a plurality of comparative data. In this, measured data may be used and/or already calculated data may be further processed.

Both the reference source and the algorithm may be the result of using artificial intelligence. Thus, machine learning tools, e.g. based on artificial neural networks, may have been used to generate the provided geometric data, in one of the ways known to the skilled person.

The geometric data may have been achieved by evaluating measurement results on limb stumps of a plurality of patients. For this purpose, for example, the actual values of the respective limb stump may have been measured, such as the cross-sections of the limb stump at a distance of, for example, 6 cm, 9 cm and 12 cm from a reference point or reference cross-section. These measurement results may be compared with the measurement results of the same patient obtained at later or still later wearing times, for example after 3 months and after 6 months. If the actual data of the patient who is to be fitted by the orthopedic technician, or of his limb stump is known, the reference source may be used to determine what the geometric data will be at later wearing times (e.g. after 3 months or after 6 months). The empirical values collected from a patient collective may therefore be used to predict what the shape of the limb stump of the specific patient P fitted with a prosthesis, for example, at the time of creation, will be at later wearing times, in particular at the reference points considered, e.g. the measuring points.

Optionally, the aforementioned reference source and/or the aforementioned algorithm are based on actual data or measured data and provide or output data that often deviates in terms of size from this, for example as geometric data.

In some embodiments of the method according to the present invention, at least 30 days, preferably at least 90 days, in particular at least 180 days, lie between the time of creation of the prosthetic shaft or the time of creation of the control file for manufacturing it on the one hand and the first wearing time of the prosthetic shaft on the other hand.

In certain embodiments, it is irrelevant whether, for example, a prosthetic shaft produced according to the present invention is actually worn at the first wearing time. Rather, it is important that it was manufactured so that it can be worn at the first wearing time, in particular of course with the highest possible wearing comfort on that day. This increased wearing comfort may be the aim of the method according to the present invention.

In several embodiments of the method according to the present invention, the geometric data additionally encompasses data that will co-determine the shape of the limb stump at at least a second wearing time. This data may take into account a change in the limb stump over time, for example the change in its dimensions (e.g., due to swelling or shrinking). In this case, the second wearing time is after the first wearing time, with at least 3 days, preferably at least 10, 20, 30, 60, 90 days, in particular at least 180 days, 12 months or 24 months, lying between the two wearing times.

In some embodiments of the method according to the present invention, a prosthetic shaft, an inner or outer shaft and/or at least one extension is also created in the step of creating. Alternatively or additionally, a control file containing control data may also be created. In these embodiments, the prosthetic shaft and/or the corresponding control file is based on the geometric data measured and/or calculated with the method according to the present invention and which will co-determine the shape of the limb stump at at least a second wearing time as described above.

It is therefore possible by the present invention to determine both data for the first wearing time as well as, preferably at the same time, that is e.g. on the same day, in the same session, during the same visit of the patient at the orthopedic technician (or vice versa), data for a second wearing time and/or further wearing times and/or to create a prosthetic shaft, inner or outer shaft or extension for the first wearing time and for the further wearing time(s).

In certain embodiments, an extension is an addition or supplement to the prosthetic shaft, an accessory, a built-in element, an inner shaft, or the like.

In some embodiments, the extension is provided to be arranged inside the outer shaft, the inner shaft, or the prosthetic shaft in general, e.g., at or from the first or further wearing time. It may preferably be given to the patient P to take home on its creation day, wherein the day of creating may correspond to the day of determining.

In several embodiments of the method according to the present invention, the inner shaft serves to receive the limb stump or at least sections thereof. In this, it is intended to be received in turn, at least in sections thereof, in an interior of the outer shaft.

In some embodiments of the method according to the invention, the extension of the prosthesis shaft is an inlay, a pad, a pressure insert, a compression insert, a stocking with different wall thicknesses or a double-walled stocking with at least one insert that is inserted between its layers.

The extension is not adjustable in some embodiments. It may optionally be depressible; it may optionally be elastic. It is not adjustable in some embodiments; for example, it may optionally be non-inflatable, not connected to a pump, not have lines or be connected thereto, and/or not be variable by the patient.

In some embodiments, the prosthesis or the prosthetic shaft does not comprise a device for actuating the extension, for example a mechanical device provided for this purpose such as an actuator, a pump, a fluid reservoir and/or lines provided for actuating the extension.

In several embodiments, the computer system is configured to be in signal communication with, or is in signal communication with, a manufacturing machine.

In some embodiments, the computer system is part of a manufacturing machine or vice versa.

In several embodiments, e.g., of the method according the present invention, the manufacturing machine for creating the prosthetic shaft or sections thereof is a printer, a 3D printer, a molding device, a milling machine, a rapid prototyping device, a CNC milling machine, a CAD milling machine, a thermoforming device, or an injection device. Here, the manufacturing machine is optionally configured to create the prosthetic shaft, inner shaft, outer shaft, or extension (or respective sections thereof) based on control signals. These control signals may in turn have been created by the method according to the present invention.

In some embodiments, the inner shaft and outer shaft are joined together, for example, by joining methods such as gluing, riveting, and the like.

The inner shaft may be dimensionally stable. It may be non-elastic. Its shape may, without wanting or needing to destroy it, be dimensionally stable or solid, at least under normal conditions of use for it or for the prosthetic shaft according to the present invention. The inner shaft may consist of or comprise carbon fibers.

The inner shaft may consist of the same material as the outer shaft or comprise the same material as the outer shaft.

The inner shaft can have an individualized circumferential shape. The individualization may consist of adapting its shape to a circumferential contour of the outer shaft.

The outer shaft may have a first strength or elasticity (expressed, for example, as total strength or total elasticity, or as average total strength or total elasticity, for example in the direction of greatest extension of the circumferential section). The inner shaft may have a second strength or elasticity that is higher than or equal to the first, that is, than that of the outer shaft, using the same procedure for measuring the strength or elasticity.

In several embodiments, the outer shaft surrounds the inner shaft only in sections, while in others it surrounds the inner shaft completely.

In certain embodiments, the inner shaft is preferably at least one of: one-piece, closed circumferential, seamlessly circumferential, without doubling in the sense of wrinkling, without step, without gap and/or slit, no elastic liner. In some embodiments, this also applies to the outer shaft.

In some embodiments, the outer shaft, the inner shaft and/or the extension(s) comprise at least one connection device by which at least two of the aforementioned components may be connected to each other.

The connection device may encompass or consist of at least one Velcro connection, an adhesive connection and/or a screw connection.

In several embodiments, the extension is not or does not comprise a pumping system.

In some embodiments, the outer shaft and/or the inner shaft are not flexible.

In several embodiments, the outer shaft and/or the inner shaft do not have a lacing system or a tensioning system that would serve to change the volume surrounded by the respective shaft or to change the diameter of the respective shaft or shaft section.

In some embodiments of the kit according to the present invention, a first element of the group, consisting of a prosthetic shaft, an inner or outer shaft or at least one extension of the prosthetic shaft, was created based on data or geometric data, which preferably co-determine the shape of the limb stump at the first wearing time. In addition, a second element of the group was created based on data or geometric data, which preferably co-determine the shape of the limb stump at the second wearing time.

Optionally, at least any element of this group was created at the time of creation, based on the available actual data of the patient P, which reflect or co-determine the shape of the limb stump of this patient at the time of creation. Such actual data may, for example be or have been measured directly on the limb stump. It does not correspond to the determined data or geometric data.

In some embodiments, the step of determining the geometric data does not comprise (or does not correspond to) any of the steps that lead to obtaining measurement data that may be obtained when measuring the limb stump, at least not in order to already create on this basis the prosthetic shaft, outer shaft, inner shaft or extension or control signals to be used at the first wearing time and created for this purpose according to the present invention. Steps leading to the obtaining of measurement data lead in particular to data obtained by measurement by measuring tape, by scanning, by laser measurement or laser scanning, by ultrasound measurement or ultrasound scanning, by determination of the proportions of solid tissue (bone) in relation to the proportions of soft tissue (fat, edema, connective tissues, muscles).

In several embodiments, the step of determining also encompasses considering actual data or measured data, referred to herein as measurement data. Obtaining the measured data, however, is optionally not part of the method.

In some embodiments, the method according to the present invention comprises particularly no ultrasonic measurement.

Determining, in several embodiments, is understood to mean reading out, estimating, predicting, and/or specifying.

In some embodiments, the method according to the present invention does not encompass a scanning step, a sonographic step, and/or a creation of a bone model.

In several embodiments, the method according to the present invention does not encompass consideration of a so-called reduction measure (RM). One speaks of a reduction measure when the orthopedic technician determines, for example, from the circumference of the thigh and from a constriction measurement, a dimension by which the dimensions of the shaft to be manufactured by the orthopedic technician must deviate from the dimensions determined on site on patient P, so that the shaft manufactured by the orthopedic technician provides sufficient support for patient P when using said shaft and when it is loaded with the patient's body weight. The reduction measurement is therefore taken into account by the orthopedic technician for dimensioning and designing the shaft of the prosthesis, with the aim of ensuring that the prosthetic shaft is as optimally as possible adapted to the measured limb stump and its properties at the time of its first use. Taking into account a reduction measurement thus serves to compensate for inadequacies that occur in connection with the measurement of the limb stump before the patient uses the prosthesis for the first time.

In several embodiments, the method also encompasses creating control signals or a control file with control signals, for creating a prosthetic shaft respectively, although this is not done based on the determined geometric data, but based on measured actual data. The actual data reflect the shape of the limb stump at the time of creation, while the data or geometric data describe or approximate the shape of the limb stump at one or more wearing time(s) lying more or less far after the creating time.

In some embodiments, the geometric data is approximated data that deviates from data that would have been measurable or was measured on patient P at the creating time or at a particular wearing time.

In several embodiments, the geometric data is not data of the specific patient P and is not data collected from the patient, but rather data collected from a patient collective or using a patient collective, or data calculated based thereon.

When it is said herein that the geometric data will co-determine the shape of the limb stump at a future wearing time, this means in several embodiments that there is a reasonable assumption that this will be the case. This assumption may be based on empirical values. It may be based on the fact that, based on the specific shape of the stump at the creating time and values determined, for example, from patient collectives, it can be assumed that the limb stump with sufficient experience will exhibit the geometric data at the targeted wearing time, such as a predicted circumference at a predetermined height, for instance measured at a predetermined distance from, for example, an immovable bony structure. It is not certain whether this will happen.

One or more of the advantages mentioned above or below may be achieved by some or all of the embodiments according to the present invention.

The limb stump is subject to daily volume fluctuations, as well as to partly significant volume changes in the months following the amputation, initially due to postoperative edema and scarring and later due to muscular atrophy.

It is desirable that the prosthetic shaft fits as if freshly fitted to the limb stump both at the moment of creating it or fitting (the creating time) and at later times (the relevant wearing time) and still provides the required stability even after muscular atrophy, especially when standing and walking. It is obvious that, for example, a prosthetic outer shaft, which must provide considerable stability to fulfill its task, cannot adapt to such a change in the cross-section of the limb stump due to its strength.

The present invention advantageously serves to provide rapid adjustment or adaptability of the prosthetic shaft to a change in shape of the limb stump. The adjustment may be conveniently performed by the wearer himself by changing the accuracy of fit of his prosthesis in a few simple steps. For example, the wearer only has to insert the extension or the inner shaft into the prosthetic shaft and fix it there, if provided (screwing, clicking in, gluing, etc.), which, based on the data determined for later use, namely for use at the first (or a further) wearing time, may already have been manufactured ahead of time at the time of creation.

The present invention is in the following exemplarily explained based on the accompanying drawings, in which identical reference numerals denote the same or similar components. The following applies in the partially highly simplified figures:

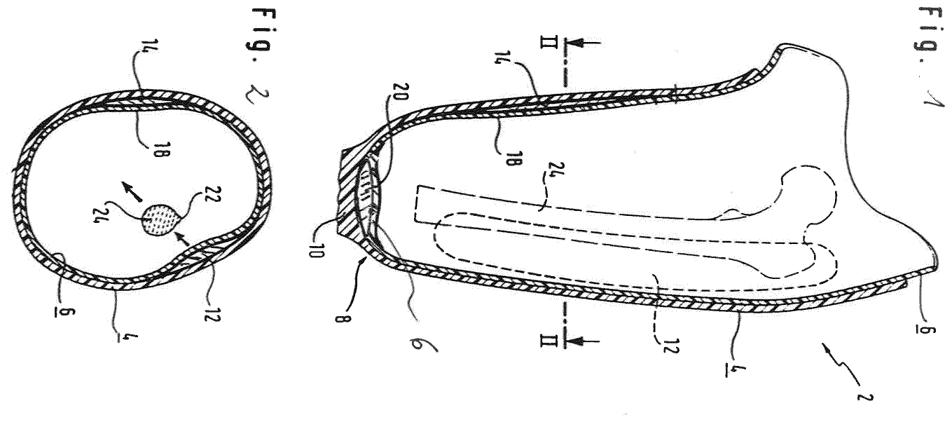

FIG. 1 shows a prosthetic shaft as part of a thigh prosthesis being only partially shown with several extensions according to the present invention in a longitudinal section;

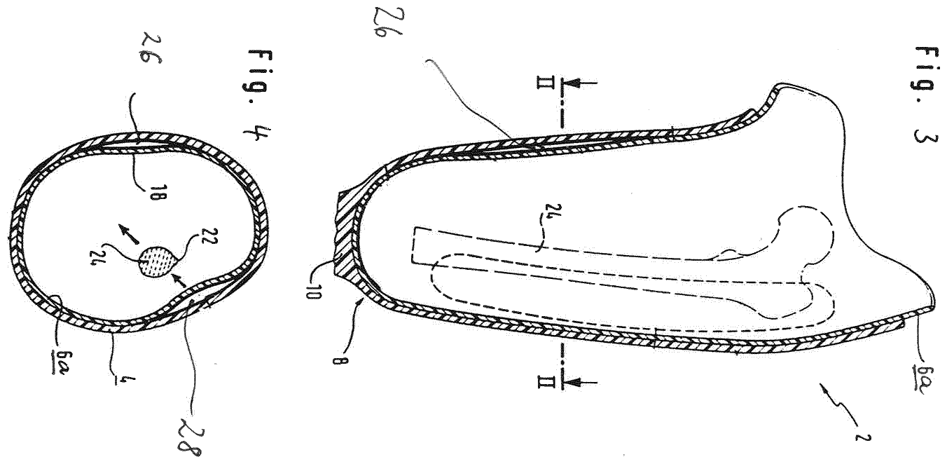

FIG. 2 shows a cross-section of the thigh prosthesis of FIG. 1;

FIG. 3 shows an inner shaft according to the present invention as part of a thigh prosthesis, which is only partially shown in a longitudinal section;

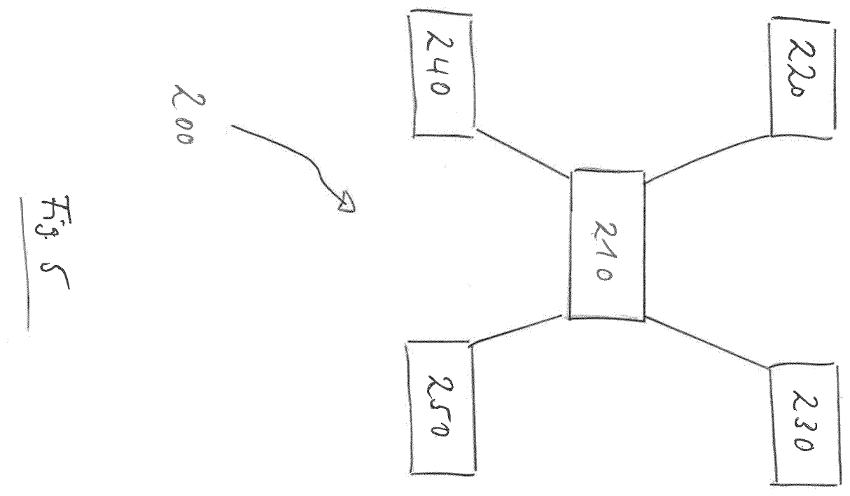

FIG. 4 shows a cross-section of the thigh prosthesis of FIG. 3;

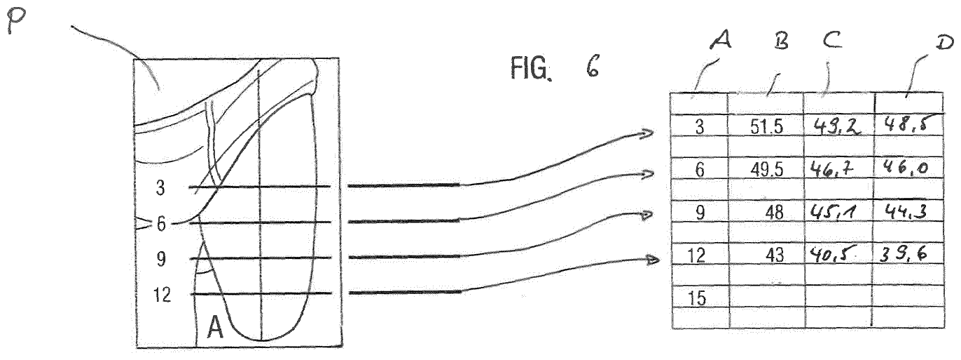

FIG. 5 shows an exemplary embodiment of the computer system according to the present invention; and

FIG. 6 shows, schematically, a reference source for use in the method according to the present invention.

FIG. 1 shows an outer shaft 4 as part of a prosthetic shaft 2 of a thigh prosthesis being only partially shown. The relatively stiff, shell-shaped outer shaft 4 receives in its interior a preferably comparatively flexible inner shaft 6 which is inserted removably and which is individually adapted to the limb stump of the patient P.

The optionally closed distal end 8 of the outer shaft 4 is followed by a column-like component 10 leading to the mechanical knee joint (not shown in FIG. 1).

Unlike prostheses of this type as known from the prior art, here—e.g. between the longitudinally extending walls of the outer shaft 4 and the inner shaft 6—e.g. two extensions 12 and 14 according to the present invention are arranged, which may each press the wall 18 of the inner shaft 6 inwards by their inner wall in the relevant areas in order to achieve a local reduction in the internal volume of the shaft.

An optional, further such extension 20 according to the present invention is located on the outside of the inner shaft 6 at its distal end.

More precisely, the extensions 12 and 14 are optionally arranged here in the dorso-lateral area following an edge 22 of the thighbone (femur) 24 (indicated by dashed lines) or in the medial-distal area. In this, the extension 12, extending from proximal to distal, optionally has an elongated shape, whereas the extension 14 optionally has a rather round shape.

As indicated by the arrows in the cross-section through the prosthetic shaft 2 of FIG. 1 shown in FIG. 2, the femur 24 intentionally undergoes a more or less strong adduction as a result of the extension 12. This allows the abduction, that usually occurs in transfemoral amputees some time after the amputation, to be corrected. In addition, the extensions 12 and 14 allow the shaft volume to be reduced and provide the residual limb with increased surface adhesion in the shaft, here: in the inner shaft 6. This surface adhesion in turn makes it possible, with the aid of the optional extension 20, to restore a desired residual limb end contact after swelling has subsided and, if necessary, after atrophy processes.

The extensions 12, 14 and 20 have been given together with the outer shaft 4 to the patient P, on whose limb stump the outer shaft 4 was adapted on the day of its manufacture (that is, for example, at the time of creation).

The use of the extensions 12, 14, 20 was not necessary on the day of transferring the prosthesis with the outer shaft 4, nor would it have provided the patient P with increased wearing comfort. According to the present invention, however, it had already been determined on or before the day of transfer (e.g., at the time of determination) how some of the data or geometric data of the limb stump would in all likelihood change in the foreseeable future (i.e., at the first wearing time). Up to a day, referred to herein as the first wearing time, the limb stump had changed due to muscular remodeling and possibly a reduction in swelling such that the outer shaft 4 produced at that time could no longer fit optimally. Patient P can independently correct the deviation between the changed shape of his limb stump and the unchangeable shape of the outer shaft 4 of his prosthesis by inserting the change in the shape (geometric data) of his limb stump that is expected in his case. In the present example, he only has to insert or use the extensions 12, 14 and 20 as already envisaged by the orthopedic technician at the time of determination and, if necessary, secure them against slipping within the prosthetic shaft 2. In this way, he can restore the desired accuracy of fit for his prosthesis without having to visit the orthopedic technician again and without expert knowledge.

FIG. 3 shows a second embodiment of the prosthetic shaft 2 in longitudinal section. FIG. 4 in turn shows a cross-section thereof.

Unlike what is shown in FIG. 1, the prosthetic shaft 2 has no extensions 12, 14 or 20. The muscular remodeling and also the decrease in possible post-operative edema are compensated for by the special design of the inner shaft 6a, which differs fundamentally from the inner shaft 6 of FIG. 1 and FIG. 2.

At the points at which in the embodiment of FIGS. 1 and 2 exemplary extensions 12 and 14 were provided to compensate for muscular remodeling with the aim of exerting pressure on the femur 24 in the direction of the arrow, in the embodiment shown in FIG. 3 and FIG. 4, the stiff inner shaft 6a shaped in a special way takes over or adopts this function. Its rigidity results in the formation of empty spaces 26 and 28. They have the shape of the extensions 14 and 12 of FIGS. 1 and 2, respectively. The rigidity of the inner shaft 6a of FIG. 3 allows it to remain form-stable while still exerting the desired pressure on the thigh stump.

The inner shaft 6a is, so to speak, a shaft from the retort: Its dimensions are not based on the dimensions that the orthopedic technician measured on the limb stump in order to fit the patient P with a prosthesis. Rather, its dimensions are based on data predicted into the future or geometric data, of or based on which it was assumed at the time of the fitting that the limb stump would assume or adopt them later and which were thus determined before or at the time of creation.

In the embodiment shown in FIGS. 3 and 4, it is therefore assumed in the present example that the inner shaft 6a is already the second inner shaft, i.e. an inner shaft that was intended to be worn only or starting from the first wearing time. It is assumed that the patient was fitted by the orthopedic technician with an inner shaft (not shown in the figures) which was placed inside the outer shaft 4 with continuous contact to the inside thereof. It is further assumed that this original inner shaft no longer fitted optimally at a first wearing time, e.g. weeks or months after being provided by the orthopedic technician, which is why it was replaced by the inner shaft 6a, shown in FIGS. 3 and 4, while retaining the original outer shaft 4.

FIG. 5 shows a computer system 200 according to the present invention.

The computer system 200 optionally comprises a calculation device 210, a reference source 220, an input device 230, an output device 240, and/or a manufacturing machine 250, respectively. The aforementioned units 220, 230, 240, and 250 are each optional and may be connected to or integrated with the calculation device 210. They may be in one-way or two-way signal communication with the calculation device 210. They may be interconnected in any manner. Each of these connections may be wired or wireless.

The calculation device 210 may serve to determine the geometric data. For this purpose, it may make use of an optional reference source 220 in which reference data may be stored. For example, by specifying the actual dimensions measured at the time of creation, optionally supplemented by other data such as the age, weight, mobility classification (1 to 4), physical activity, etc. of the patient P, which may optionally be entered by the input device 230, by simply associating this data with empirical values of already existing geometric data, which the limb stump is likely to assume at certain times in the future (referred to herein as wearing times) may be output. The output may be done by the output device 240, e.g. in the form of a notification on a display or as a printout for the orthopedic technician. In addition to or instead of an output, control signals (individually or as part of a control file) may be transmitted to the manufacturing machine 250. The desired component, for example the inner shaft or the extension, or a section or parts thereof, can be produced on it, optionally automatically. The indication and/or control signals may encompass information as to where, for example, produced extensions 12, 14, 20 are to be placed in the prosthetic shaft 2.

FIG. 6 shows an example of how provided data or geometric data is used.

On the left in FIG. 6, a limb stump of patient P, who is only partially shown, may, when being measured, have the measurement results stated in the table on the left in FIG. 6. Column B shows the measurement results obtained on the stump and indicates the respective measured circumference (in cm) at a distance of, for example, 6 cm, 9 cm and 12 cm from a reference point or reference cross-section (column A). The values in column B are also regarded as actual values. They were measured at the positions of the limb stump specified in column A before the prosthetic shaft 2 was created.

When examining a large number of patients with comparable limb stumps, values were measured in advance of the method according to the invention which indicate the cross-sections of the limb stump, e.g. 6 cm, 9 cm and 12 cm, at later, defined wearing times. For example, the numerical values in column C indicate which circumferential values were determined for the collective at the positions specified in column A at a first wearing time, for instance after 3 months after creation of the prosthesis, possibly based on or related to determined actual values. At the same time, they indicate which measured values the limb stump of patient P would presumably assume, since they have already applied to a sufficiently large collective, for example by reflecting the changes in the measured values observed for the collective over 3 months.

Column D gives circumferential values, for which one may assume, due to the previous measurements on the above-mentioned patient collective, that the actual limb stump shown on the left in FIG. 6 will also assume these (or very similar) values/dimensions at the positions specified in column A at the second wearing time, after about 6 months.

FIG. 6 shows a reference source on the right. The columns C and D thereof show which geometric data a limb stump, which at the time of initial fitting with a prosthesis (e.g. at the time of determination) has the values of column B at the positions specified in column A, will in all probability have at the, herein exemplarily considered, first and second wearing times, namely the values of columns C and D. Reading them out may represent a determination in the sense of the present invention.

If patient P is a diabetic, the values in columns C and D may be determined from a collective that also consisted of diabetics.

Furthermore, the values in columns C and D can already take into account how large the values in column B are. If the specific patient P had shown greater actual values than those noted in column B, the values C and D could also have been greater.

FIG. 6 serves as an example. The present invention is not limited to considering the circumference as a geometric datum. The use of other data is supplementary or alternative, in combination with one another or alone, likewise encompassed by the present invention.

Instead of a reference source, geometric data may be determined based on a present set of data at the moment of determination.

Although the present invention is described or discussed herein in a number of passages and in particular on the basis of the exemplary figures using the example of the limb stump of a lower extremity (thigh, lower leg, foot), the present invention is by no means limited to the fitting of a limb stump of the lower extremity. According to the invention, what is described herein also applies without restriction to the fitting of the upper extremity (upper arm, lower arm, hand) as well as to the products proposed for fitting, such as prosthetic shaft, inner shaft, outer shaft and extensions.

### LIST OF REFERENCE NUMERALS

2 prosthetic shaft

4 outer shaft

5 inlet opening

6 inner shaft

7 slot or slit

8 distal end of the outer shaft

10 column-like or columnar component

12 Extension or accessoire

14 Extension or accessoire

18 wall

20 extension or accessoire

22 edge

24 femur

200 computer system

210 calculation device

220 reference source

230 input device

240 output device

250 manufacturing machine

1. A method for manufacturing or for planning the manufacturing of a prosthetic shaft, an inner shaft, an outer shaft and/or an extension of the prosthetic shaft, wherein the prosthetic shaft is provided for receiving a limb stump of a patient P,
encompassing the steps:
determining geometric data or providing geometric data, wherein the geometric data helps determine the shape of the limb stump at at least a first wearing time of the prosthetic shaft, inner shaft, outer shaft, or of the extension;
and either
creating, based on the determined geometric data, the prosthetic shaft, the inner or outer shaft, or the extension, or creating sections thereof, based on the geometric data,
or
creating a control file having control signals upon which a manufacturing machine may execute steps for creating the prosthetic shaft, the inner shaft, outer shaft or the extension,
wherein said creating takes place at a time of creating prior to the first wearing time, and wherein the geometric data is not data measured on the patient P.

2. The method according to claim 1, wherein the geometric data is predicted data, data of variable dimensions of the limb stump, data which is subject to, or caused by, post-operative changes over time and changes caused by a previous surgical operation, and/or data which does not represent or reflect the shape of the limb stump at the time of determining the data or at the time of creating, and/or it is not actual data and/or measured data of the patient P.

3. The method according to claim 1, wherein the determining of the geometric data, which is predicted data, takes into account patient data which reflects the, or at least one, in particular momentary, health condition or finding of the patient P at the time of the determining and/or at a past time or takes into account patient data which was collected from a collective of patients having this health condition or finding.

4. The method according to claim 1, wherein the geometric data is or encompasses the result of an estimation, a readout from a reference source and/or a calculation based on an algorithm.

5. The method according to claim 1, wherein at least 3 days, preferably at least 10, 20, 30, 60, 90 days, in particular at least 180 days, 12 months or 24 months lie between the creation time and the first wearing time.

6. The method according to claim 1, wherein the geometric data additionally also encompasses data which will co-determine the shape of the limb stump at at least a second wearing time which is after the first wearing time, wherein at least 3 days, preferably at least 10, 20, 30, 60, 90 days, in particular at least 180 days, 12 months or 24 months lie between the first wearing time and the second wearing time.

7. The method according to claim 1, wherein in the step of creating, a prosthetic shaft, an inner shaft, an outer shaft and/or at least one extension or a corresponding control file for the manufacturing machine is also created based on the geometric data which will co-determine the shape of the limb stump at least at the second wearing time.

8. The method according to claim 1, wherein the inner shaft serves to receive at least portions of the limb stump and in turn is provided to be at least partially received in an interior of the outer shaft.

9. The method according to claim 1, wherein the extension is an inlay, a pad, a pressure insert, a compression insert, a stocking with different wall thicknesses or a double-walled stocking with at least one insert inserted between its layers.

10. The method according to claim 1, wherein the manufacturing machine is a printer, a 3D printer, a casting device, a milling machine, a rapid prototyping device, a CNC milling machine, a CAD milling machine, a thermoforming device, or nowadays also an injection device, configured to create the prosthetic shaft, the inner shaft, the outer shaft or the extension or sections thereof based on the control signals.

11. A prosthetic shaft, an inner shaft, an outer shaft and/or an extension for the prosthetic shaft, manufactured by the method according to claim 1.

12. A kit with at least two elements from a group consisting of prosthetic shaft, inner shaft, outer shaft or at least of one extension, each manufactured according to the method of claim 1.

13. The kit according to claim 12, wherein at least a first element of the group was created based on geometric data which co-determines the shape of the limb stump at the first wearing time, and wherein at least a second element of the group was created based on geometric data which co-determines the shape of the limb stump at the second wearing time.

14. A computer system programmed to carry out the method according to claim 1.

15. A digital storage medium, in particular a floppy disk, CD or DVD or EPROM, with electronically readable instructions, configured in order to configure a control device and/or a closed-loop control device into a control device and/or a closed-loop control device with which the method according to claim 1 may be executed.

16. A computer program product with a program code stored on a machine-readable carrier, configured in order to configure a control device and/or a closed-loop control device into a control device and/or a closed-loop control device with which the method according to claim 1 may be executed.

17. A computer program with a program code for configuring a control device and/or a closed-loop control device into a control device and/or a closed-loop control device with which the method according to claim 1 may be executed.
